(19) United States
(12) Patent Application Publication
(10) Pub. No.: US 2014/0066739 A1
(43) Pub. Date: Mar. 6, 2014

(54) SYSTEM AND METHOD FOR QUANTIFYING OR IMAGING PAIN USING ELECTROPHYSIOLOGICAL MEASUREMENTS

(71) Applicants: Bin He, Arden Hills, MN (US); Huishi Zhang, Minneapolis, MN (US)

(72) Inventors: Bin He, Arden Hills, MN (US); Huishi Zhang, Minneapolis, MN (US)

(21) Appl. No.: 14/012,618

(22) Filed: Aug. 28, 2013

Related U.S. Application Data
(60) Provisional application No. 61/694,497, filed on Aug. 29, 2012.

(51) Int. Cl. A61B 5/00; G01R 33/48; A61B 5/0478; A61B 5/04

(52) U.S. Cl. CPC A61B 5/4824; A61B 5/04012; A61B 5/7203; A61B 5/04008; G01R 33/4806; A61B 5/0478; A61B 5/742
USPC 600/377; 600/544; 600/409; 600/411; 600/300; 600/383

(57) ABSTRACT

A method for computing a quantitative metric indicative of pain experienced by a subject using an electrophysiological signal detection system is provided. Electrophysiological data are acquired from a subject with the electrophysiological signal detection system. Modulations in the acquired electrophysiological data that are associated with pain experienced by the subject during the acquisition of the electrophysiological data are identified. A quantitative metric indicative of the pain experienced by the subject is computed by processing the identified modulations in the acquired electrophysiological data.

### STATEMENT REGARDING FEDERALLY SPONSORED RESEARCH

This application is based on, claims priority to, and incorporates herein by reference in its entirety U.S. Provisional Application Ser. No. 61/694,497 filed on Aug. 29, 2012, and entitled “System and Method for Quantifying and Image Pain from Electrophysiological Measurements.”

### STATEMENT REGARDING FEDERALLY SPONSORED RESEARCH

This invention was made with government support under EB006433 and EB007920 awarded by the National Institutes of Health. The government has certain rights in the invention.

### BACKGROUND OF THE INVENTION

The field of the invention is systems and methods for electrophysiology. More particularly, the invention relates to systems and methods for quantifying and imaging pain using electrophysiological measurements.

Pain represents the most important cause of physician consultation in the United States, and more than 30 million people are suffering from chronic or recurrent pain. Patients who suffer from chronic pain or recurrent pain usually take mediations, such as analgesics, to reduce or eliminate their pain. However drug therapy planning is highly influenced by the subjective pain ratings of the patients. Thus, there is a clinical need to develop noninvasive approaches to quantitatively assess pain severity levels. The availability of quantitative pain severity assessment technology will have a significant impact on the clinical management of pain, and will provide physicians with the means to objectively guide and optimize drug therapies.

Functional imaging of brain networks associated with pain processing is of vital importance to better understand the mechanisms of brain function in addition to aid the development of new pain-relief therapy. The pain response in the brain is a complex process that involves multiple cortical brain regions, such as primary and secondary somatosensory cortices, anterior cingulated cortex, and insular cortex. Recent advancement in neuroimaging techniques suggests the possibility to map the brain structure and networks that involve pain processing.

Electroencephalography (“EEG”) is a noninvasive technique that is widely used to probe neurological disorders with high temporal resolution. Few attempts have been made to use EEG to map the active brain regions in pain patients. Functional magnetic resonance imaging (“fMRI”) measures the hemodynamic brain response and can be used to image active brain regions with high spatial resolution. Studies have shown that fMRI is a useful tool to delineate the brain regions associated with pain processing.

Recent studies from simultaneous EEG and fMRI recording have suggested that the EEG response to the pain may be correlated with the fMRI response, and both EEG and fMRI could be used to image the brain pain processing regions, such as the primary somatosensory cortex and anterior cingulated cortex. However, the EEG analysis and fMRI analysis in the studies were performed separately and only the induced pain in healthy subjects was investigated.

Most current studies about pain processing by the brain were targeted at induced pain, not spontaneous pain. Only a few studies about pain processing were related to the more clinically-relevant spontaneous pain due to the difficulties in comparing the painful and pain-free conditions of spontaneous pain. The MEG sources of spontaneous pain were previously studied in a patient with phantom limb pain. The EEG sources of spontaneous pain were studied in neurogenic pain patients at the group-level analysis. The spontaneous pain in patients with chronic back pain was also studied using fMRI.

However, to our knowledge, no prior EEG source imaging study has been performed on spontaneous pain to study the brain pain processing at the individual level. In addition, to our knowledge, no prior study on spontaneous pain has been performed with the multimodal functional imaging integrating EEG and fMRI. It remains important to noninvasively quantify and image the brain processing in clinically-relevant spontaneous pain of chronic pain patients.

### SUMMARY OF THE INVENTION

The present invention provides a method for computing a quantitative metric indicative of pain experienced by a subject using an electrophysiological signal detection system. Electrophysiological data are acquired from a subject with the electrophysiological signal detection system. Modulations in the acquired electrophysiological data that are associated with pain experienced by the subject during the acquisition of the electrophysiological data are identified. A quantitative metric indicative of the pain experienced by the subject is computed by processing the identified modulations in the acquired electrophysiological data. For instance, an absolute or relative power change in a spectral band associated with the identified modulations may be computed. The present invention also provides a means of imaging and localizing the brain networks involved in pain processing from EEG measurements acquired from an array of electrode sensors placed over the scalp of a subject. The EEG signals recorded from the multiplicity of locations may be processed using signal processing algorithms such as the independent component analysis to extract independent components corresponding to the pain in a subject. Such EEG component corresponding to the pain is then imaged back to the brain space to reveal neuronal groups that are involved in the pain processing. The present invention further provides a method of imaging and localizing neural networks involved in the pain processing of a subject from simultaneous EEG and fMRI measurements, to co-localize and image the pain networks.

In accordance with one aspect of the invention, a system is provided for generating at least one quantitative metric indicative of pain experienced by a subject. The system includes at least one sensor configured to acquire electrophysiological data from a subject including information about operating of a brain of the subject. The system also includes a processor coupled to the at least one sensor to receive the electrophysiological data. The processor is configured to identify modulations in the electrophysiological data that are associated with pain experienced by the subject and compute a quantitative metric indicative of the pain experienced by the subject by processing the identified modulations in the electrophysiological data.

In accordance with another aspect of the invention, a method is provided for computing a quantitative metric indicative of pain experienced by a subject using an electrophysiological signal detection system. The method includes acquiring electrophysiological data from a subject with the electrophysiological signal detection system and identifying modulations in the acquired electrophysiological data that are associated with pain experienced by the subject during the acquisition of the electrophysiological data. The method also includes computing a quantitative metric indicative of the pain experienced by the subject by processing the identified modulations in the acquired electrophysiological data.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of a method for computing a quantitative metric of pain and producing an image indicative of the source of neuronal signals associated with pain;

FIG. 2 is a block diagram of an example of an EEG system that may be used to acquire electrophysiological data; and

FIG. 3 is a block diagram of an example of an MRI system that may be used to acquire functional MRI data.

FIG. 4 is a graph showing data in a human subject experienced “pain” and “no pain” by means of spinal cord stimulator. The EEG quantification revealed a frequency modulation in the beta band. ‘In pain’ condition refers to patient pain rating of 8 on 0 to 10 scale. ‘No pain’ condition refers to patient rating of 0.

FIG. 5 is a graph showing a correlation between quantified pain from EEG and the pain rating by a human subject.

FIG. 6 is an example image revealing brain regions involved in pain processing as estimated from the EEG collected during “in pain” condition in a patient revealing activation in anterior cingulate cortex (ACC).

FIG. 7 is a graph showing alpha rhythm modulation being negatively correlated with the stimulation state: stimulus-on vs. stimulus-off conditions.

FIGS. 8A and 8B are graphs that show alpha modulation at contralateral sensorimotor electrodes vs. ipsilateral alpha modulation.

FIGS. 9A and 9B are images that show source imaging results in a subject revealing the involvement of sensorimotor area in response to the thermal stimulation using distributed source imaging (FIG. 9A) and equivalent dipole localization (FIG. 9B).

### DETAILED DESCRIPTION OF THE INVENTION

The present invention provides systems and methods for quantifying pain from electrophysiological data. Examples of electrophysiological data include measurements or recordings made using an electroencephalography (“EEG”) system, a magnetoencephalography (“MEG”) system, or other wearable or implantable electrophysiological sensors. It is an aspect of the present invention to provide systems and methods for quantifying pain from electrophysiological data using signal processing and imaging techniques to identify brain networks involved in pain generation and processing. The pain quantification and imaging results may be used to guide and optimize drug or other therapies of patients suffering from a variety of pain types.

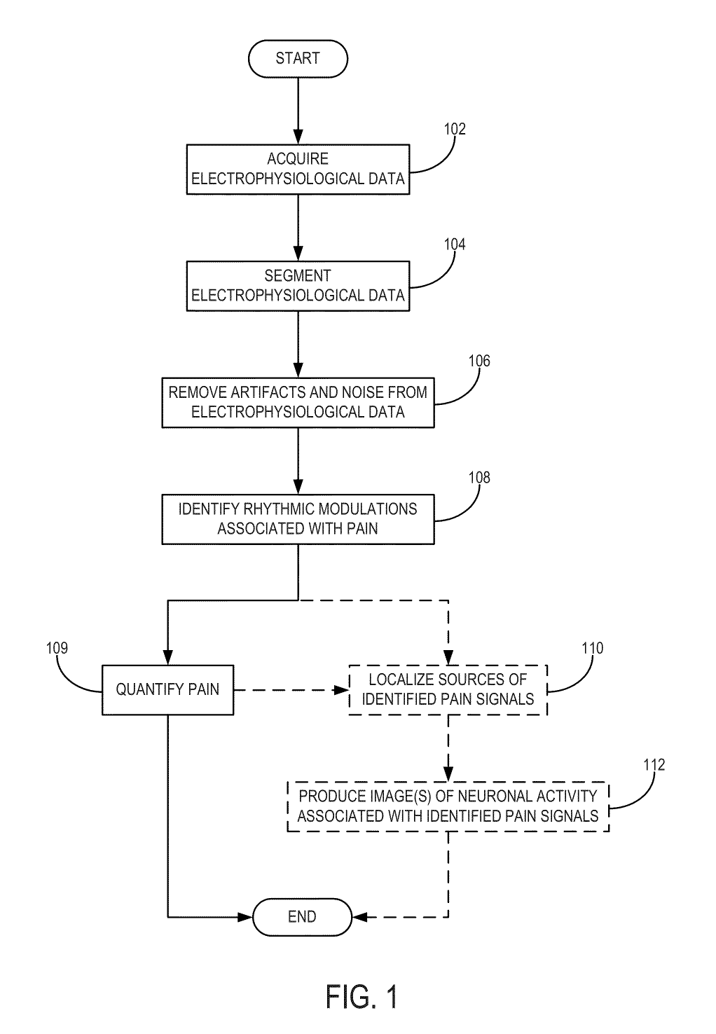

Referring now to FIG. 1, a flowchart setting forth the steps of an example of a method for quantifying and imaging pain from electrophysiological data is illustrated. The method begins with the acquisition of electrophysiological data, as indicated at step 102. This electrophysiological may be acquired by placing one or more electrophysiological sensors on, in, or near the subject in order to record electrophysiological signals generated by the subject's brain during a pain event, such as during a spontaneous pain event. The electrophysiological data may be acquired using an electrode sensor on the scalp to record EEG data, an electrode sensor over the cortex or within the brain to record intracranial EEG data, or a magnetic sensor to record MEG data, or a sensor being placed over or near the skin of the head to record an electrophysiological data. The acquisition of the electrophysiological data is done over a period of time sufficiently long to sense the pain status from which quantification and imaging can be obtained.

The acquired electrophysiological data may be segmented, as indicated at step 104, before time-frequency analysis is performed to identify and extract rhythmic modulations associated with pain. Examples of rhythmic modulations associated with pain include beta rhythm modulations, alpha rhythm modulations, theta rhythm modulations, or gamma rhythm modulations. By way of example, the acquired electrophysiological data can be divided into multiple segments that include spontaneous electrophysiological data. Multiple segments of electrophysiological data can be concatenated to form a data series sufficiently long suitable for analysis using techniques such as the independent component analysis (ICA) or principal component analysis (PCA).

As will be further described, the present invention can be used to analyze, image, and or quantify chronic or sustained pain. The particular clinical application of analyzing or attempting to determine any objective information about chronic or sustained pain is well known to be difficult. For example, when using systems, including EEG or MEG systems, to acquire data from the subject experiencing the chronic or sustained pain, the sustained signal associated with the pain can be lost or indistinguishable from noise because there may not be any periodicity or distinguishing trigger point to use to distinguish the signal associated with the pain from the noise.

However, in accordance with the present invention noise or artifacts can be removed from the electrophysiological data using band-pass filters and a blind source separation method, such as ICA, as indicated at step 106. By way of example, data segments and peripheral channels with severe muscle artifacts may also be removed from the electrophysiological data all together. ICA is a data-driven technique to separate spatiotemporal signals into components with temporal independence. An ICA algorithm such as the infomax ICA algorithm described by A. J. Bell and T. J. Sejnowski in “An information-maximization approach to blind separation and blind deconvolution,” Neural Comput, 1995; 7(6):1129-1159, can be used to decompose the spatiotemporal electrophysiological data into a time-by-space formulation:

x=QWT (1);

where x is the acquired electrophysiological data, Q is an N×N matrix of spatial distributions of the electrophysiological signals, W is an N×N diagonal scaling matrix, and T is an N×M matrix of time courses. Equation (1) can be expanded as follows:

$$x = {\sum }_{i = 1}^{N} {Q}_{i} {w}_{i} {T}_{i} ; \qquad ( 2 )$$

where Qᵢ is the i^{th} column of the spatial distribution matrix, Q; Tᵢ is the i^{th} row of the time course matrix, T; and wᵢ is the i^{th} diagonal element of the diagonal matrix, W. Equation (2) suggests that the electrophysiological data, x, can be expressed as a weighted superposition of a series of spatial distributions, Qᵢ, multiplied by associated time courses, Tᵢ, where each time course, Tᵢ, is statistically independent from the other time courses. The temporal, spectral, and spatial characteristics of the components can be used to identify and remove artifacts in the electrophysiological data, such as those due muscle movements and the like. After removing artifacts and noise, the remaining independent components can be recombined to obtain noise-free electrophysiological signals. Alternatively, an independent component corresponding to the pain process can be used for further analysis of deriving biomarkers to quantify pain or imaging and localizing the pain networks.

A fast Fourier transformation (“FFT”) based analysis can then be performed on all recorded channels to identify and extract rhythmic modulations associated with pain, as indicated at step 108. For example, the power spectral density between frequencies f₁ and f₂ can be computed on the concatenated signal using FFT. An example of f₁ and f₂ can be 1 Hz and 50 Hz, respectively. Total power and relative percentage power pertaining to each spectral band can be computed individually. The discrete Fourier transformation of the previously denoised signal, x, is given by:

Y=FFT(x) (3).

The total power in any given spectral band can be computed as the sum of the squared Y values between frequency bounds f₁ and f₂:

$$P = {\sum }_{{f}_{1}}^{{f}_{2}} {Y}^{2} . \qquad ( 4 )$$

The percentage power is the total power of the given spectral band divided by the total power from f₁ and f₂. By way of example, rhythmic components in theta (4-8 Hz), alpha (8-13 Hz), beta (13-30 Hz), gamma (30-Hz) and other bands can be analyzed. Power spectrogram can be calculated on all recorded channels as well as non-noisy components.

Biomarkers quantifying pain can be derived from processed rhythmic modulation as in step 109. By selecting the appropriate spectral band, both the relative and total power change can be used to quantify the severity of pain. By way of examples, the following indices can be computed to find neurophysiological correlates of pain, including 1) absolute power change, P_{ab}; 2) frequency percentage change, fPₚₑᵣ; 3) spatial percentage change, sPₚₑᵣ; and 4) temporal modulation Pₘ.

The absolute power change P_{ab} can be used to measure absolute power changes between pain vs. no pain or different levels of pain. It can be calculated by subtracting total power in a given band between two status (e.g. baseline no pain vs. pain). The frequency percentage change FPₚₑᵣ measures percentage power changes over the entire frequency band between two conditions (e.g. pain vs. no pain). It can be calculated by normalizing P_{ab} to the integral of the entire frequency band in a given subject. The spatial percentage change SPₚₑᵣ measures percentage power changes at a given region over the entire brain. The temporal modulation Pₘ measures the temporal modulation of power across time. Pₘ was the Pearson's cross correlation between power fluctuation and the time course representing different pain levels. These indices can be calculated on each non-noise independent components. Independent components can be then ranked according to the degree of correlation between each index with subject pain rating or other measures.

Other metrics that may be derived from the spectral distribution of ICA-processed electrophysiological signals can also be used to quantify pain. For instance, biomarkers from the source signals, including the signal strength at the region of interest during appropriate frequency components, can be used to quantify pain. The corresponding spatial map of the selected independent components will be subject to source imaging analysis, as will be described below.

Additionally or alternatively, having identified the electrophysiological signals associated with pain, the sources of these signals can be identified and imaged, as indicated at step 110. Brain sources representing neuronal activation due to pain can be localized and imaged by solving the inverse problem of EEG or MEG. By way of example, a distributed source model can be used to this end. In such a model, a number of current dipoles with unconstrained or constrained orientations can be positioned within the brain volume or occupy the gray matter. A cortical current source model may also be used, in which it makes use of a number of current dipoles with either unconstrained orientations or orientations that are perpendicular to the cortical surface. The number of dipoles in these distributed source models may be in the range of 5,000-10,000. Alternatively, a single moving dipole model or multiple dipole source models may also be used, with each dipole representing one focused area of brain activity.

By way of example, in the EEG or MEG forward model, the spatiotemporal electrophysiological data, x, can be related to the underlying brain activity, S, through the following linear system:

x=LS+B (5);

where x is an n×t signal matrix, in which n is the number of sensors and t is the number of time points; S is an m×t source matrix, in which m is the dimension of source space; B is an n×t noise matrix; and L is an n×m lead field matrix. The lead field matrix, L, can be determined to solve the forward problem of the EEG or MEG. For example it can be obtained using a boundary element method (“BEM”), as described by M. Fuchs, et al., in “An improved boundary element method for realistic volume-conductor modeling,” IEEE Trans Biomed Eng., 1998; 45(8):980-997; a finite element method (“FEM”); a finite difference method; or another suitable numerical method. In the BEM model, the head volume conductor can be separated into three conductive layers: the brain, the skull, and the skin, with suitable conductivities. Alternatively, the BEM model can be separated into four conductive layers: the brain, the skull, the skin, and the cerebrospinal fluid (“CSF”). These forward head models using BEM or FEM or other methods may be constructed based upon the anatomic imaging data of a subject using for example an MRI. Alternatively a generic head model derived from a large number of subjects may also be used. A three-dimensional distributed source model can be used to model the brain source distribution, which includes around five to ten thousands equivalent current dipoles with unconstrained orientations uniformly positioned within the three-dimensional brain volume or grey matter.

The electrophysiological data, x, can be decomposed into independent components as shown in Eqn. (2). Given the forward modeling of the lead field matrix, L, spatiotemporal brain sources can be estimated from the electrophysiological data, x, by solving an inverse problem of Eqn (5) as follows:

S=L⁺x (6);

where L⁺ is the general inverse of the lead field matrix L. Substituting Eqn. (6) into Eqn. (2), the spatiotemporal estimation can be rewritten as:

$$\begin{matrix}\begin{matrix}\hat{S} = {L}^{+} {\sum }_{i = 1}^{{N}_{s}} {w}_{i} {Q}_{i} {T}_{i} \\ = {\sum }_{i = 1}^{{N}_{s}} ( {L}^{+} {Q}_{i} ) {w}_{i} {T}_{i} \\ = {\sum }_{i = 1}^{{N}_{s}} {\hat{S}}_{i} {w}_{i} {T}_{i} ;\end{matrix} & ( 7 )\end{matrix}$$

where Ŝᵢ=L⁺Qᵢ is the independent component source distribution of the i^{th} independent component, and

$${\sum }_{i = 1}^{{N}_{s}} {\hat{S}}_{i} {w}_{i} {T}_{i}$$

is the linear combination of pain signal components in the source space, which can be seen as an inverse process of ICA. The independent component source distribution, Ŝᵢ, of each pain component can be computed using a low resolution electromagnetic tomography (“LORETA”) method, as described by R. D. Pascual-Marqui, et al., in “Low resolution electromagnetic tomography: a new method for localizing electrical activity in the brain,” Int J Psychophysiol, 1994; 18:49-65. Alternatively, the independent component source distribution, Ŝᵢ, of each pain component can be computed using other EEG/MEG distributed imaging algorithms, such as minimum norm estimate (“MNE”) algorithms; variants of MNE algorithms, such as weighted MNE algorithms; L−p norm algorithms, such as L−1 norm algorithms; sub-space scanning algorithms, such as MUSIC and RAP-MUSIC algorithms; FINE algorithms; or dipole source localization algorithms.

Given the reconstructed dynamic source signal, Ŝ, the pain signal sources can be estimated as averaged activity during a time duration, or as the relative change of the signals as compared with no pain condition as follows:

$$\frac{{S}_{p} - {S}_{n}}{{S}_{n}} ; \qquad ( 8 )$$

where Sₚ refers to the averaged source signal during a pain condition and Sₙ, refers to a source signal during no pain condition. Alternatively, the pain network may be delineated and imaged from one independent component after ICA decomposition. In such a case, the independent component corresponds to a pain condition.

Sources of pain signals may also be localized by solving a moving dipole localization problem from multi-channel EEG or MEG data. First of all, the ICA can be used to denoise the data and extract components of interest corresponding to pain. Then the pain sources may be localized by solving the optimization problem of:

min(∥x−Ly∥ₚ) (9);

where x is denoised independent component corresponding to pain, L the lead field matrix, and y the inverse dipole solution. ∥ ∥ₚ represents p-norm for the residual of recorded and model predicted electrophysiological signals, where p can be 1, 2 or another value.

Either or both EEG-informed fMRI analysis and fMRI-constrained EEG source analysis can be performed to investigate brain networks involved in pain generation, as indicated at step 112. The EEG-informed fMRI approach can be used to convolve the temporal independent component waveform with a hemodynamic response function and to supply the convolved waveform to the GLM analysis. A reciprocal imaging approach can be used by applying the EEG-informed fMRI results to constrain the EEG source imaging. Brain networks involved with pain can be delineated and then quantified by extracting pain biomarkers based on regions-of-interest (ROI). Similarly, the brain imaging approach can be used to delineate networks involved with multiple pain conditions. For each pain condition, ROI-based analysis can be applied to quantify pain.

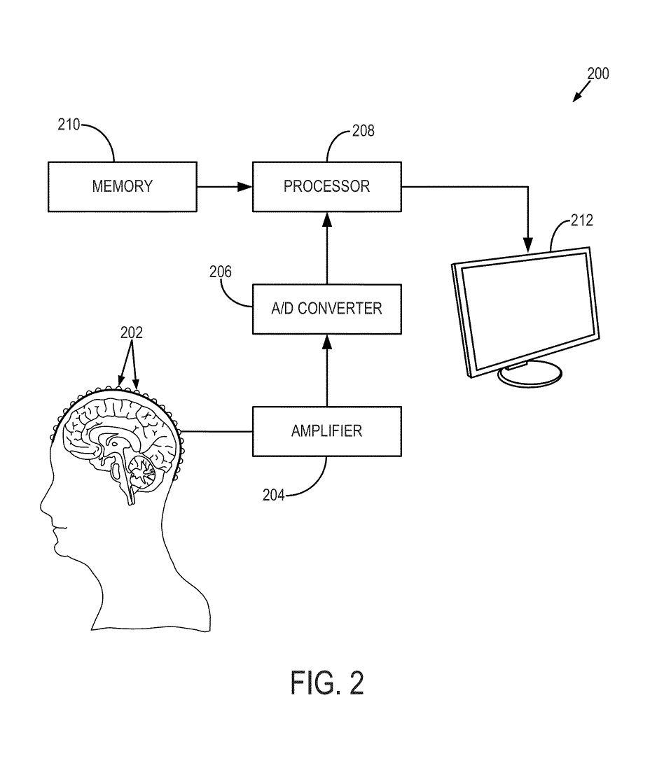

Referring now to FIG. 2, an example of an EEG system 200 that may be used to acquire electrophysiological data indicative of neuronal activity is illustrated. The electrophysiological signals measured and acquired as electrophysiological data with the EEG system 200 are acquired on a number of EEG electrodes 202, or sensors.

During measurement of neuronal activity with the EEG system, a continuous stream of voltage data representative of an electrophysiological signal is detected by the electrodes 202, which are coupled to the subject's scalp, and the acquired signals are sampled and digitized. Specifically, an amplifier 204 in communication with the electrodes 202 is used to amplify the acquired signals, after which the amplified signals are sent to an analog-to-digital (“A/D”) converter 206 that converts the signals from analog to digital format. The acquired signals can also undergo additional preprocessing in order to remove artifacts, such as those due to data collection and physiological causes. The digital signals are sent to a processor 208 that processes the signals as described in detail above. The processor 208 is also configured to store the processed or unprocessed signals in a memory 210, and to display the signals on a display 212.

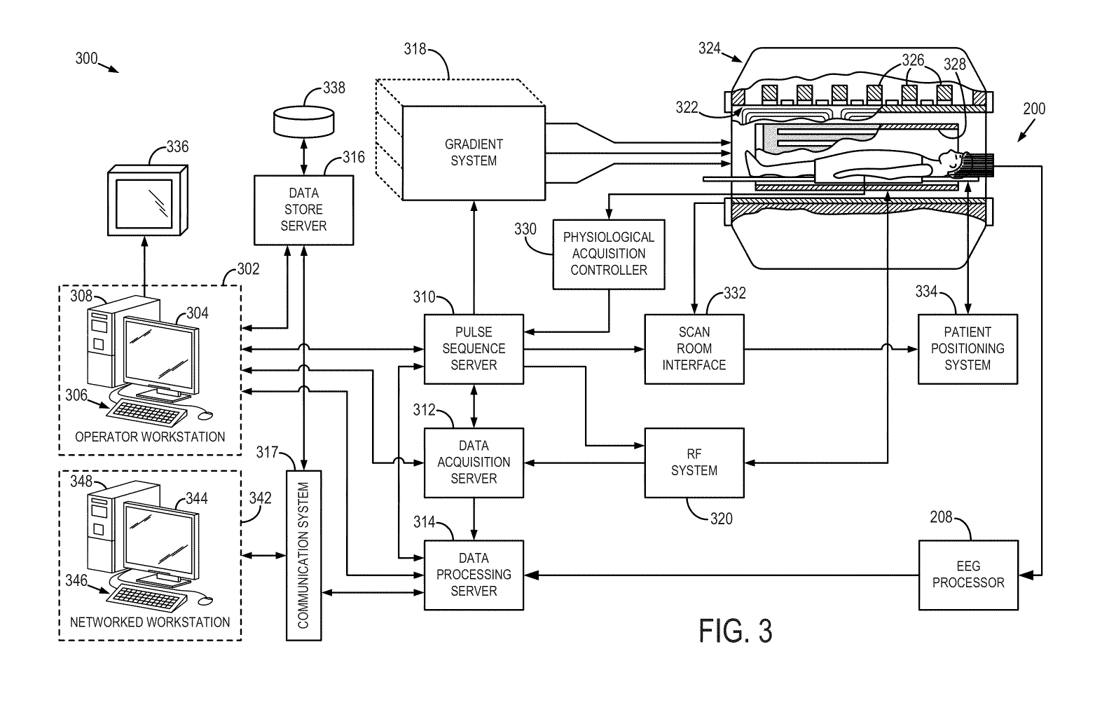

Referring particularly now to FIG. 3, an example of a magnetic resonance imaging (“MRI”) system 300 that may be used to acquire functional MRI (fMRI) images is illustrated. The MRI system 300 includes a workstation 302 having a display 304 and a keyboard 306. The workstation 302 includes a processor 308, such as a commercially available programmable machine running a commercially available operating system. The workstation 302 provides the operator interface that enables scan prescriptions to be entered into the MRI system 300. The workstation 302 is coupled to four servers: a pulse sequence server 310; a data acquisition server 312; a data processing server 314; and a data store server 316. The workstation 302 and each server 310, 312, 314, and 316 are connected to communicate with each other via a communication system 317, which may include any suitable network connection, whether wired, wireless, or a combination of both. As an example, the communication system 317 may include both proprietary or dedicated networks, as well as open networks, such as the internet.

The pulse sequence server 310 functions in response to instructions downloaded from the workstation 302 to operate a gradient system 318 and a radiofrequency (“RF”) system 320. Gradient waveforms necessary to perform the prescribed scan are produced and applied to the gradient system 318, which excites gradient coils in an assembly 322 to produce the magnetic field gradients Gₓ, G_{y}, and G_{z} used for position encoding MR signals. The gradient coil assembly 322 forms part of a magnet assembly 324 that includes a polarizing magnet 326 and a whole-body RF coil 328.

RF excitation waveforms are applied to the RF coil 328, or a separate local coil (not shown in FIG. 3), by the RF system 320 to perform the prescribed magnetic resonance pulse sequence. Responsive MR signals detected by the RF coil 328, or a separate local coil (not shown in FIG. 3), are received by the RF system 320, amplified, demodulated, filtered, and digitized under direction of commands produced by the pulse sequence server 310. The RF system 320 includes an RF transmitter for producing a wide variety of RF pulses used in MR pulse sequences. The RF transmitter is responsive to the scan prescription and direction from the pulse sequence server 310 to produce RF pulses of the desired frequency, phase, and pulse amplitude waveform. The generated RF pulses may be applied to the whole body RF coil 328 or to one or more local coils or coil arrays (not shown in FIG. 3).

The RF system 320 also includes one or more RF receiver channels. Each RF receiver channel includes an RF amplifier that amplifies the MR signal received by the coil 328 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} (10);

and the phase of the received MR signal may also be determined:

$$ϕ = {tan}^{- 1} ( \frac{Q}{I} ) . \qquad ( 11 )$$

The pulse sequence server 310 also optionally receives patient data from a physiological acquisition controller 330. The controller 330 receives signals from a number of different sensors connected to the patient, such as electrocardiograph (“ECG”) signals from electrodes, or respiratory signals from a bellows or other respiratory monitoring device. Such signals are typically used by the pulse sequence server 310 to synchronize, or “gate,” the performance of the scan with the subject's heart beat or respiration.

The pulse sequence server 310 also connects to a scan room interface circuit 332 that receives signals from various sensors associated with the condition of the patient and the magnet system. It is also through the scan room interface circuit 332 that a patient positioning system 334 receives commands to move the patient to desired positions during the scan.

In accordance with the present invention, the MRI system 300 may be used in conjunction with the EEG system 200 or an MEG or other electrophysiological system (not shown in FIG. 3). In this regard, the EEG processor 208 may be configured to communicate with the data processing server 314 or other components of the MRI system 300 to coordinate the acquisition of EEG data with the acquisition of MRI data, such as fMRI data.

The digitized MR signal samples produced by the RF system 320 are received by the data acquisition server 312. The data acquisition server 312 operates in response to instructions downloaded from the workstation 302 to receive the real-time MR data and provide buffer storage, such that no data is lost by data overrun. In some scans, the data acquisition server 312 does little more than pass the acquired MR data to the data processor server 314. However, in scans that require information derived from acquired MR data to control the further performance of the scan, the data acquisition server 312 is programmed to produce such information and convey it to the pulse sequence server 310. For example, during prescans, MR data is acquired and used to calibrate the pulse sequence performed by the pulse sequence server 310. Also, navigator signals may be acquired during a scan and used to adjust the operating parameters of the RF system 320 or the gradient system 318, or to control the view order in which k-space is sampled. The data acquisition server 312 acquires MR data and processes it in real-time to produce information that is used to control the scan.

The data processing server 314 receives MR data from the data acquisition server 312 and processes it in accordance with instructions downloaded from the workstation 302. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 314 are conveyed back to the workstation 302 where they are stored. Real-time images are stored in a data base memory cache (not shown in FIG. 3), from which they may be output to operator display 312 or a display 336 that is located near the magnet assembly 324 for use by attending physicians. Batch mode images or selected real time images are stored in a host database on disc storage 338. When such images have been reconstructed and transferred to storage, the data processing server 314 notifies the data store server 316 on the workstation 302. The workstation 302 may be used by an operator to archive the images, produce films, or send the images via a network to other facilities.

The MRI system 300 may also include one or more networked workstations 342. By way of example, a networked workstation 342 may include a display 344; one or more input devices 346, such as a keyboard and mouse; and a processor 348. The networked workstation 342 may be located within the same facility as the operator workstation 302, or in a different facility, such as a different healthcare institution or clinic.

The networked workstation 342, whether within the same facility or in a different facility as the operator workstation 302, may gain remote access to the data processing server 314 or data store server 316 via the communication system 317. Accordingly, multiple networked workstations 342 may have access to the data processing server 314 and the data store server 316. In this manner, magnetic resonance data, reconstructed images, or other data may be exchanged between the data processing server 314 or the data store server 316 and the networked workstations 342, such that the data or images may be remotely processed by a networked workstation 342. This data may be exchanged in any suitable format, such as in accordance with the transmission control protocol (TCP), the internet protocol (IP), or other known or suitable protocols.

### EXAMPLE 1

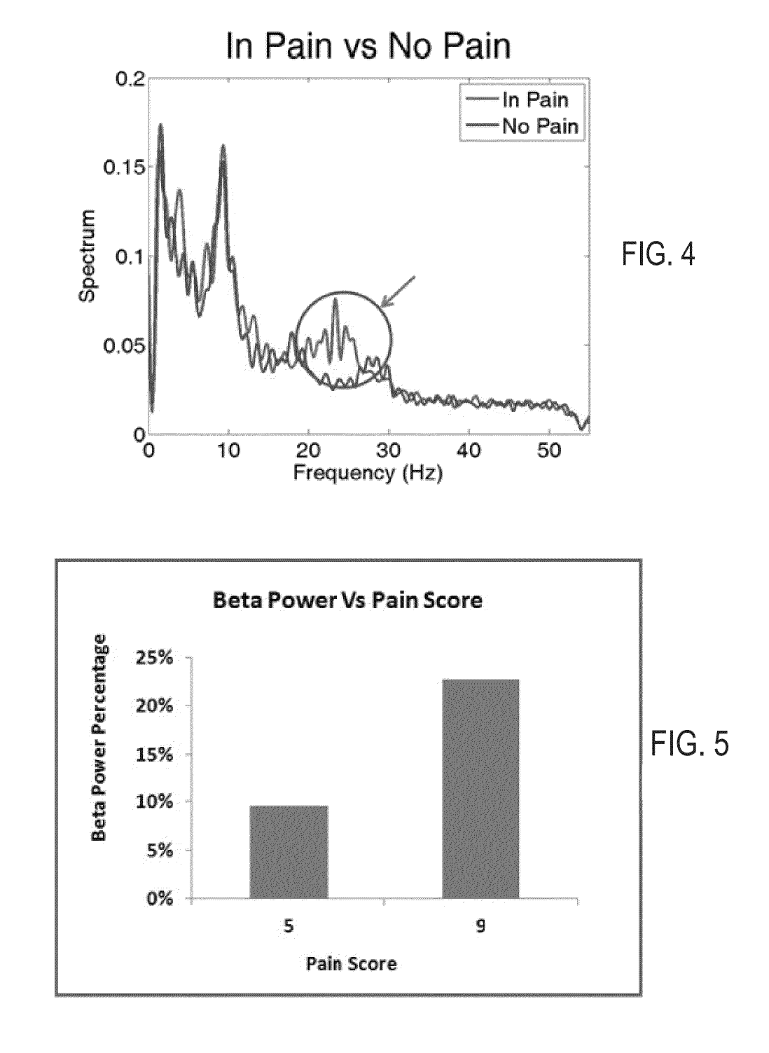

Applying the present invention to a patient population identified rhythmic activity changes in the beta band that are correlated with patient's pain perception. In two patients implanted with spinal cord stimulator (SCS), 128-channel dense array scalp EEGs were recorded during “in pain” condition after turning off SCS and “no pain” condition with SCS modulation. FIG. 4 shows an example of beta modulation in a patient comparing “in pain” vs. “no pain”. FIG. 4 indicates the ability of the present invention to quantitatively derive biomarkers reflecting pain in a patient, through measurement and processing of noninvasive EEG over the scalp. FIG. 5 shows a quantification result of beta power (9% and 23%) in relation to subject pain rating (“5” and “9”), as derived from the beta modulation of EEG in a subject.

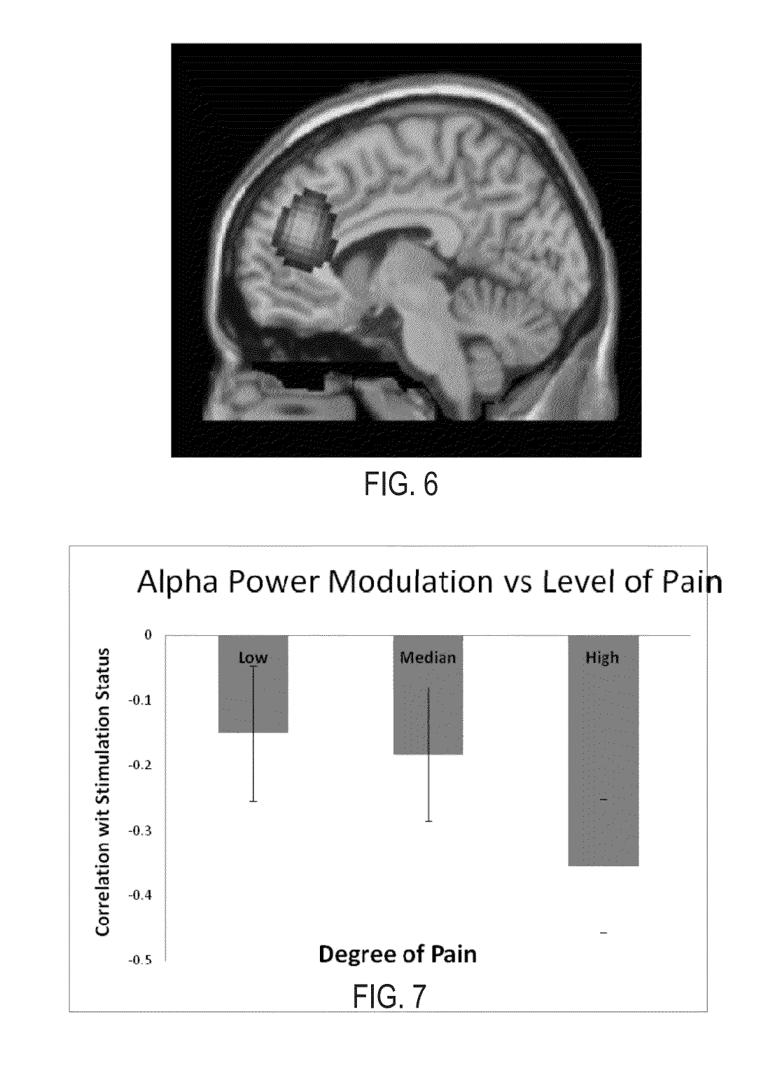

FIG. 6 shows noninvasive EEG source imaging of brain network during in pain condition, indicating anterior cingulate cortex is involved in pain after the SCS was turned off. In this example, 128 ch EEG were processed using ICA to extract independent component corresponding to pain condition. The source distribution was then estimated using the LORETA inverse algorithm and visualized together an anatomic MRI.

Using the aforementioned ICA-based source localization method, brain areas associated with pain perception and processing were identified and imaged. By way of example, the power spectral density between 1 to 125 Hz may be computed on the concatenated signals using FFT. Total power and relative percentage power pertaining to each rhythmic band may be computed individually. It is contemplated that a consistent positive correlation between a rhythmic band relative power and patients' pain scores will be found, indicating a pain condition.

### EXAMPLE 2

Applying the present invention to another embodiment revealing its ability to quantify and image pain in a group of healthy human subjects. Each subject experienced a sustained painful stimulus using a thermal stimulator with the thermode placed on the dorsal side of their left or right wrist. Depending on individual tolerance, the temperature of the thermode was kept at a range from 40 to 47 for 30 seconds during the stimulus-on condition. During this session, subjects experienced a sustained painful heat. Then the temperature dropped to and stayed at 32° C. for 60 seconds during the stimulus-off condition. During this session, subjects experience a light touch sensation. Each trial was repeated 10 times. Thermode was moved slightly after each recording session to avoid sensitization or habituation on the same stimulation site. EEG signal was collected with a 60-channel EEG system.

Raw EEG was down-sampled to 250 Hz and first high pass filtered at 1 Hz. 60 Hz power line noise was removed with a notch filter. A continuous 25 seconds of EEG data from both each 30-second stimulation-on and stimulation-off portion were segmented. The 25 seconds segments were selected such that they started after 4.5 seconds of each stimulation start or end. This was to avoid any transient effects due to rapid heating or cooling. Ten pairs of segmented data of both stimulation-on and stimulation-off were concatenated sequentially according to the actual temporal order.

Alpha rhythm power in the independent component with high sensorimotor alpha percentage was found to be negatively correlated with the stimulation state: stimulus-on vs stimulus-off conditions (FIG. 7). This counter modulation is most pronounced at the highest intensity of pain. The group level correlation coefficient was found to be −0.4±0.15 between the alpha rhythm and the stimulation status, when the pain rating was at the highest level.

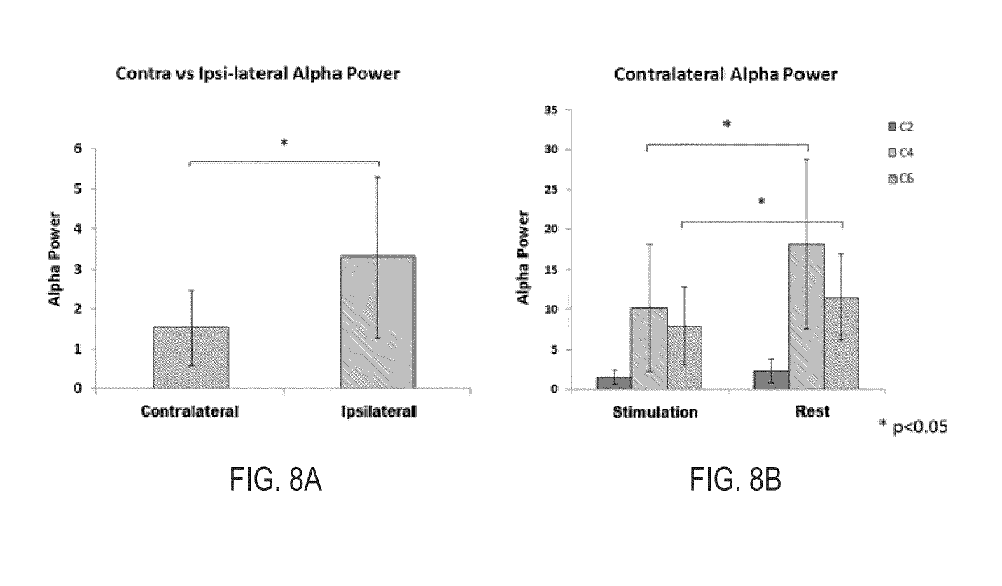

Frequency analysis was performed on both contralateral and ipsilateral electrodes in the sensorimotor region. In the temporal aspect, it is found that the alpha activity at the contralateral sensorimotor electrodes was suppressed during the painful stimulation-on condition by comparing to stimulation-off condition using the t statistical testing (FIG. 8A) (p<0.05). The alpha power at the contralateral area was also found to be statistically smaller than the alpha power of ipsilateral brain region during the painful thermal stimulation (FIG. 8B) (p<0.05).

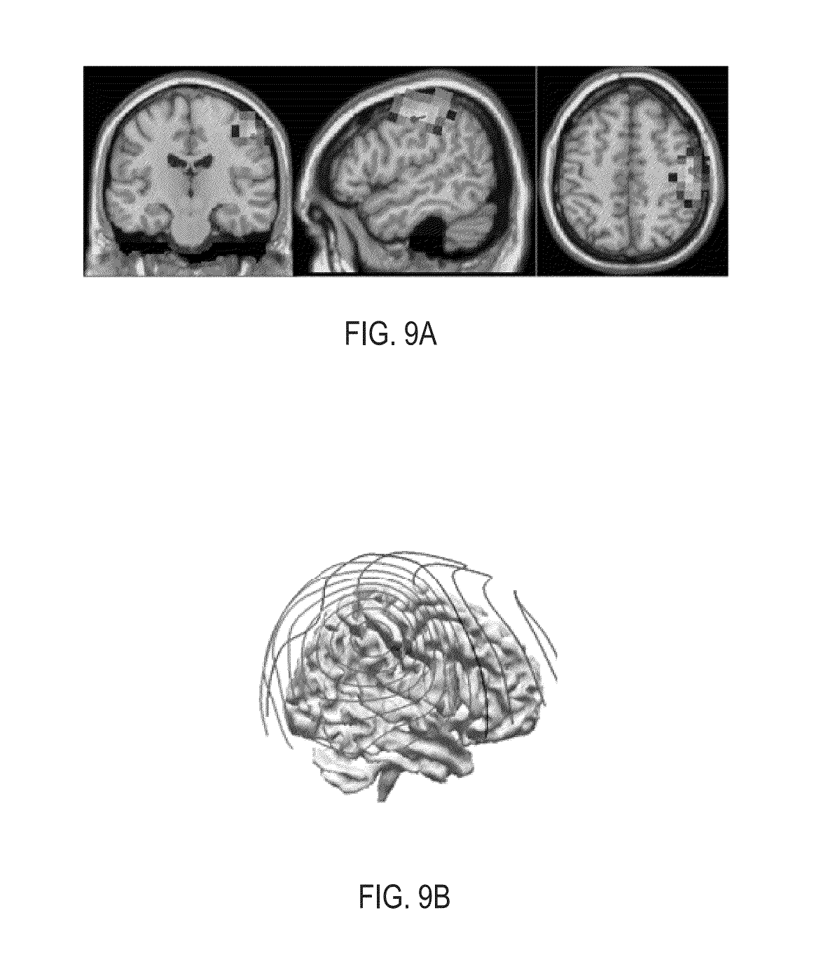

Neurological sources that were most responsive to stimulation, thus pain, were inversely estimated using the spatial map of selected independent components. These components showed tight coupling the power fluctuation that corresponds to stimulation status. Localization results yielded distributed current density with the peak at the sensorimotor area (FIG. 9A). A single equivalent dipole was also localized at the sensorimotor cortex corresponds to the site of simulation i.e. left wrist (FIG. 9B).

As thermal stimulation intensity is correlated with pain perception, this example provides experimental evidence that the present invention can quantify sustainable pain as well as imaging the brain regions involved in pain processing.

The present invention has been described in terms of one or more preferred embodiments, and it should be appreciated that many equivalents, alternatives, variations, and modifications, aside from those expressly stated, are possible and within the scope of the invention.

1. A system for generating at least one quantitative metric indicative of pain experienced by a subject, the system comprising:
at least one sensor configured to acquire electrophysiological data from a subject including information about operating of a brain of the subject;
a processor coupled to the at least one sensor to receive the electrophysiological data and configured to:
identify modulations in the electrophysiological data that are associated with pain experienced by the subject; and
compute a quantitative metric indicative of the pain experienced by the subject by processing the identified modulations in the electrophysiological data.

2. The system of claim 1 wherein the processor is further configured to separate the electrophysiological data into components with temporal independence.

3. The system of claim 2 wherein the processor is further configured to analyze at least one of temporal, spectral, and spatial characteristics of the components to identify and remove artifacts in the electrophysiological data.

4. The system of claim 3 wherein the processor is further configured to recombine the components remaining after removing the artifacts and is configured to use the recombined components to compute the quantities metric.

5. The system of claim 2 wherein the processor is further configured to separate the electrophysiological data into a time-by-space formulation given by:
x=QWT
where x is the acquired electrophysiological data, Q is an N×N matrix of spatial distributions of the electrophysiological signals, W is an N×N diagonal scaling matrix, and T is an N×M matrix of time courses.

6. The system of claim 1 wherein the processor is further configured to express the electrophysiological data, x, as a weighted superposition of a series of spatial distributions, Qᵢ, multiplied by associated time courses, Tᵢ, where each time course, Tᵢ, is statistically independent from the other time courses.

7. The system of claim 6 wherein the processor is further configured to decompose the spatiotemporal electrophysiological data into a time-by-space formulation given by:
$$x = {\sum }_{i = 1}^{N} {Q}_{i} {w}_{i} {T}_{i}$$
where Qᵢ is the i^{th} column of the spatial distribution matrix, Q; Tᵢ is the i^{th} row of the time course matrix, T; and wᵢ is the i^{th} diagonal element of the diagonal matrix, W.

8. The system of claim 1 wherein the processor is further configured to use at least one of temporal, spectral, and spatial characteristics of the electrophysiological data to remove artifacts in the electrophysiological data.

9. The system of claim 8 wherein the artifacts in the electrophysiological data include noise due muscle movements of the subject.

10. The system of claim 1 wherein the quantitative metric includes at least one of total power in a given spectral band of the electrophysiological data, percentage power of the given spectral band of the electrophysiological data, and signal strength of the electrophysiological data at a given region of interest.

11. The system of claim 1 wherein the at least one sensor for at least one of an electroencephalography (“EEG”) system and a magnetoencephalography (“MEG”) system.

13. The system of claim 1 wherein the processor is further configured to receive functional magnetic resonance imaging (fMRI) data of the subject and correlate the quantitative metric with the fMRI data.

14. A method for computing a quantitative metric indicative of pain experienced by a subject using an electrophysiological signal detection system, the steps of the method comprising:
a) acquiring electrophysiological data from a subject with the electrophysiological signal detection system;
b) identifying modulations in the acquired electrophysiological data that are associated with pain experienced by the subject during the acquisition of the electrophysiological data in step a); and
c) computing a quantitative metric indicative of the pain experienced by the subject by processing the identified modulations in the acquired electrophysiological data.

15. The method as recited in claim 14 in which step c) includes computing at least one of an average power and a relative power change in a spectral band associated with the identified modulations.

16. The method as recited in claim 14 further comprising localizing and imaging sources of the identified modulations using a source imaging algorithm.

17. The method as recited in claim 14 in which step a) includes acquiring functional magnetic resonance imaging (MRI) data from the subject with an MRI system concurrently with the electrophysiological data, and in which analysis of the functional MRI data is guided using the identified modulations.

18. The method as recited in claim 14 in which step b) includes using of the independent component analysis to analyze electrophysiological data recording during pain perception.

19. The method as recited in claim 14 in which step a) includes acquiring electroencephalography signals using an electrode sensor over the skin of head or within a tissue of the head.

20. The method as recited in claim 14 in which step a) includes acquiring magnetoencephalography signals using a sensor proximate to the scalp.
